(19) United States
(12) Patent Application Publication
(10) Pub. No.: US 2025/0050118 A1
(43) Pub. Date: Feb. 13, 2025

(54) LEADLESS PACEMAKERS INCLUDING A PIEZOELECTRIC DEVICE

(71) Applicant: University of Washington, Seattle, WA (US)

(72) Inventors: Babak Nazer, Seattle, WA (US); Mohammad Malakooti, Seattle, WA (US)

(21) Appl. No.: 18/794,618

(22) Filed: Aug. 5, 2024

Related U.S. Application Data
(60) Provisional application No. 63/518,033, filed on Aug. 7, 2023.

(51) Int. Cl. A61N 1/378; A61N 1/05; A61N 1/375

(52) U.S. Cl. CPC A61N 1/3785; A61N 1/056; A61N 1/37512; A61N 1/3756

(57) ABSTRACT

An example leadless pacemaker includes a housing including at least one outer surface and at least one energy storage device. The leadless pacemaker also includes at least one electrode coupled to the at least one energy storage device and configured to generate electrical pulses that are delivered to one or more chambers of the heat. The leadless pacemaker further includes at least one piezoelectric device disposed on at least a portion of the outer surface of the housing. The piezoelectric device is electrically coupled to the energy storage device. The piezoelectric device is configured to be generate electrical energy responsive to pressure changes in the heart.

### BACKGROUND

Pacemakers are implanted medical devices that are configured to generate electrical pulses and provided the electrical pulses to the heart. The electrical pulses applied to the heart may help regulate the electrically conductive system of the heart. Pacemakers may help treat a variety of heart-related conditions, such as bradyarrhythmia, atrio-ventricular block, and sinus node dysfunction.

### SUMMARY

Embodiments disclosed herein related to energy-harvesting leadless pacemakers (“leadless pacemakers”) and methods of making and using the same. In an embodiment, an energy-harvesting leadless pacemaker is disclosed. The energy-harvesting leadless pacemaker includes a housing including at least one outer surface. The housing includes at least one energy storage device. The energy-harvesting leadless pacemaker also includes at least one electrode coupled to the at least one energy storage device. The at least one electrode is configured to generate electrical pulses that are delivered to one or more chambers of a heart. Furthermore, the energy-harvesting leadless pacemaker includes at least one piezoelectric device disposed on at least a portion of the at least one outer surface of the housing. The at least one piezoelectric device is electrically coupled to the at least one energy storage device. The at least one piezoelectric device is configured to generate electrical energy responsive to pressure changes in the heart.

In an embodiment, a method of using an energy-harvesting leadless pacemaker is disclosed. The method includes disposing the energy-harvesting leadless pacemaker in a heart. The energy-harvesting leadless pacemaker includes a housing comprising at least one outer surface. The housing includes at least one energy storage device. The energy-harvesting leadless pacemaker also includes at least one electrode coupled to the at least one energy storage device. The at least one electrode is configured to generate electrical pulses that are delivered to one or more chambers of a heart. Furthermore, the energy-harvesting leadless pacemaker includes at least one piezoelectric device disposed on at least a portion of the at least one outer surface of the housing. The at least one piezoelectric device is electrically coupled to the at least one energy storage device. The at least one piezoelectric device is configured to generate electrical energy responsive to pressure changes in the heart. The method also includes generating the electrical energy with the at least one piezoelectric device as the heart beats.

In an embodiment, a method of making an energy-harvesting leadless pacemaker is disclosed. The method includes disposing at least one piezoelectric device on at least one outer surface of a housing. The housing including at least one energy storage device. The energy-harvesting leadless pacemaker also includes at least one electrode coupled to the at least one energy storage device. The at least one electrode is configured to generate electrical pulses that are delivered to one or more chambers of a heart. Furthermore, the energy-harvesting leadless pacemaker includes at least one piezoelectric device disposed on at least a portion of the at least one outer surface of the housing. The at least one piezoelectric device is electrically coupled to the at least one energy storage device. The at least one piezoelectric device is configured to generate electrical energy responsive to pressure changes in the heart.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a leadless pacemaker, according to an embodiment.

FIG. 1B and 1C are cross-sectional views of the leadless pacemaker of FIG. 1A taken along planes 1B-1B and IC-IC, respectively.

FIG. 1D is an enlarged cross-sectional view of the piezoelectric device taken from the box 1D shown in FIG. 1C.

FIG. 1E is an isometric view of a method of forming the leadless pacemaker when the piezoelectric material and the insulator material exhibit the spiral arrangement, according to an embodiment.

FIG. 2 is a cross-sectional view of a leadless pacemaker, according to an embodiment.

FIG. 3 is a cross-sectional view of a leadless pacemaker, according to an embodiment.

FIG. 4 is a cross-sectional view of a portion of a piezoelectric device with a sealant disposed thereon, according to an embodiment.

### DETAILED DESCRIPTION

Embodiments disclosed herein related to leadless pacemakers and methods of making and using the same. An example leadless pacemaker includes a housing including at least one outer surface and at least one energy storage device. The leadless pacemaker also includes at least one electrode coupled to the at least one energy storage device and configured to generate electrical pulses that are delivered to one or more chambers of the heart. The leadless pacemaker further includes at least one piezoelectric device disposed on at least a portion of the outer surface of the housing. The piezoelectric device is electrically coupled to the energy storage device. The piezoelectric device is configured to generate electrical energy responsive to pressure changes in the heart.

During use, the leadless pacemaker may be disposed in a heart of the individual. For example, the leadless pacemaker may be surgically implanted in the heart of the individual via the femoral vein using a thin catheter. In a particular example, the leadless pacemaker may be disposed in the right ventricle of the heart.

After disposing the leadless pacemaker in the heart, the leadless pacemaker may be exposed to changes in pressure caused by the beating of the heart. These pressure changes may cause deformation of the piezoelectric device. The deformation of the piezoelectric device causes the piezoelectric device to generate electrical energy. The electrical energy generated by the piezoelectric device may be provided to the energy storage device of the leadless pacemaker, thereby partially charging the energy storage device. The electrical energy provided to the energy storage device increases the lifespan of the leadless pacemaker compared to a substantially similar leadless pacemaker that does not include the piezoelectric device.

The leadless pacemakers disclosed herein are an improvement over conventional pacemakers. One example of conventional pacemakers includes transvenous pacemakers. Transvenous pacemakers including a housing that is spaced from the heart of the individual, such as in the chest of the individual near the skin of the individual, and one or more leads extending from the housing. The housing includes an energy storage device and one or more leads extending from the housing. The leads may extend from the housing, through the veins of the individual, and to the heart of the individual. The location of the housing in the chest of the individual allows the energy storage device to be wirelessly charged using a charger external to the individual.

It has been found that transvenous pacemakers have several issues associated therewith. For example, the leads extending through the veins of the individual increase the risk of infection and blood clots in the individual. The leads of the transvenous pacemaker may also break during use thereby requiring surgery to repair the transvenous pacemaker. Additionally, the surgery used to insert the transvenous pacemaker in the individual and/or repair a broken lead is relatively intrusive thereby requiring long recovery times and resulting in significant scarring. Furthermore, individuals having the transvenous pacemakers may have activity restrictions because of the relatively intrusive surgery, to limit the likelihood that the lead breaks, and because of the location of the transvenous pacemaker in the chest. As such, transvenous pacemakers may not be used with individuals who are susceptible to infections (e.g., individuals who previously had infections caused by a transvenous pacemaker), are susceptible to blood clots, do not desire invasive surgeries, or desire limited activity restrictions.

Conventional leadless pacemakers were designed to overcome several of these issues associated with transvenous pacemakers. Leadless pacemakers do not include lead(s) since the leadless pacemakers are disposed directly in the heart of the individual. As such, leadless pacemakers do not have some of the issues associated with leads. For example, compared to transvenous pacemakers, leadless pacemakers are less likely to cause infection or blood clots in the individual and are less likely to break during use. Furthermore, leadless pacemakers may be introduced into the heart of the individual via a thin catheter inserted into the femoral vein. As such, the surgical procedure used to insert the leadless pacemaker into the individual may be a less invasive surgical technique than the surgical procedures used to insert the transvenous pacemaker into the individual. Furthermore, leadless pacemakers have fewer activity restrictions than transvenous pacemakers, for example, due to the less invasive surgical techniques use to dispose the leadless pacemakers in the individual.

However, conventional leadless pacemakers have several issues associated therewith. For example, it is difficult if not impossible to wirelessly charge leadless pacemakers due to the location of the leadless pacemakers in the individual. As such, the lifespan of the leadless pacemakers is generally limited to the lifespan of the energy storage device included therein, which is typically about 5 years to about 12 years. Furthermore, the heart tissue generally grows around the leadless pacemaker over time making retrieval of the leadless pacemakers difficult or impossible should the energy storage device of the leadless pacemaker run out of energy. As such, when the energy storage device runs out of energy, a new leadless pacemaker is disposed in the heart while the old pacemaker is left in the heart. The heart has limited volume to receive leadless pacemakers which limits the number of leadless pacemakers that an individual may receive. As such, leadless pacemakers may not be used with younger individuals who are likely to need multiple pacemakers during their lifespans. This is a significant downside to leadless pacemakers compared to transvenous pacemakers, which easily have their generators replaced with a minor surgical procedure, and may otherwise operate indefinitely assuming the leads continue to function well.

The leadless pacemakers disclosed herein solve several of these issues associated with conventional transvenous pacemakers and conventional leadless pacemakers. For example, the leadless pacemakers disclosed herein have the same benefits over transvenous pacemakers for at least the same reasons as conventional leadless pacemakers. Furthermore, the piezoelectric devices of the leadless pacemakers disclosed herein recharge the energy storage devices of the leadless pacemakers disclosed herein. The ability to recharge the energy storage devices of the leadless pacemakers disclosed herein increases the lifespan of the leadless pacemakers disclosed herein compared to substantially similar leadless pacemakers that do not include the piezoelectric device. The increased lifespan of the leadless pacemakers disclosed herein allow more individuals to use leadless pacemakers and minimize recurrence of surgical procedures needed to ensure that the individuals always have functioning pacemakers.

Typically, piezoelectric devices have not been used in leadless pacemakers for several reasons. For example, leadless pacemakers have strict size limitations to allow the leadless pacemakers to be disposed in the tissue of the heart without impeding operation of the heart and to allow the leadless pacemakers to be inserted into the heart using less invasive surgical procedures. It is noted that other implanted devices, such as transvenous pacemakers, do not have such strict size limitations. It was believed that disposing piezoelectric devices on conventional leadless piezoelectric devices would prevent the leadless pacemakers from being disposed in the tissue of the heart or being used with the less invasive surgical procedures. However, it has been surprisingly found that the piezoelectric devices and housing disclosed herein enable the formation of leadless pacemakers that meet the strict size limitations for leadless pacemakers. In another example, the pressure changes that the leadless pacemakers are exposed to in the heart (and, in particular, the right ventricle) are significantly less than the pressure changes in the arteries and other locations that typically receive transvenous pacemakers or similar implanted devices. It was believed that the lower pressure changes in the heart would cause the piezoelectric devices to generate insignificant energy, especially since the more efficient piezoelectric materials are not biocompatible. However, it has been surprisingly found that the piezoelectric devices disclosed herein may generate 10% or more of the energy needed to operate the leadless pacemakers which, in turn, can increase the lifespan of the leadless pacemakers by a year or more (including years), depending on the size of the energy storage device and the efficiency of the piezoelectric device.

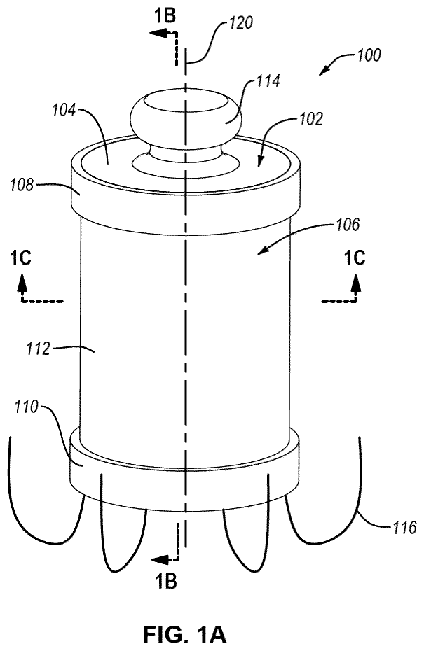

FIG. 1A is an isometric view of a leadless pacemaker 100, according to an embodiment. FIG. 1B and 1C are cross-sectional views of the leadless pacemaker 100 taken along planes 1B-1B and 1C-1C, respectively. The leadless pacemaker 100 includes a housing 102. The housing 102 includes at least one outer surface 104. The leadless pacemaker 100 also includes at least one piezoelectric device 106 disposed on at least a portion of the outer surface 104 of the housing 102.

The leadless pacemaker 100 may exhibit any suitable shape. In an example, the leadless pacemaker 100 may exhibit a generally cylindrical shape. The generally cylindrical shape of the leadless pacemaker 100 may facilitate moving the leadless pacemaker 100 through veins or through catheters while inserting the leadless pacemaker 100 into the individual. The cylindrical shape may also prevent the leadless pacemaker 100 from having edges that may cut tissue during use. That said, the leadless pacemaker 100 may exhibit any suitable shape, without limitation.

As previously discussed, the leadless pacemaker 100 may need to exhibit strict size limitations. For example, the leadless pacemaker 100 needs to exhibit a maximum lateral dimension (e.g., diameter) that allows the leadless pacemaker 100 to fit through veins during the surgery without rupturing the veins. Furthermore, the leadless pacemaker 100 needs to exhibit a length that is sufficiently small to prevent a proximal end of the leadless pacemaker 100 from entrapping in the tricuspid valve or impairing the valve's ability to fully close. In an example, the leadless pacemaker 100 may exhibit a maximum lateral dimension (e.g., diameter when the leadless pacemaker 100 exhibits a generally cylindrical shape) and a maximum length which is measured parallel to a longitudinal axis 120 of the leadless pacemaker 100. The maximum lateral dimension of the leadless pacemaker 100 may be selected to be about 3 mm to about 10 mm, such as in ranges of about 3 mm to about 5 mm, about 4 mm to about 5 mm, about 4.5 mm to about 5.5 mm, about 5 mm to about 6 mm, about 5.5 mm to about 6.5 mm, about 6 mm to about 7 mm, about 6.5 mm to about 7.5 mm, about 7 mm to about 8 mm, about 7.5 mm to about 9 mm, or about 8 mm to about 10 mm. The maximum length of the leadless pacemaker 100 may be selected to about 15 mm to about 60 mm, such as in ranges of about 15 mm to about 25 mm, about 20 mm to about 30 mm, about 25 mm to about 35 mm, about 30 mm to about 40 mm, about 35 mm to about 45 mm, about 40 mm to about 50 mm, about 45 mm to about 55 mm, or about 50 mm to about 60 mm.

The housing 102 forms an interior structure of the leadless pacemaker 100. At least a portion of the housing 102 may be a rigid structure that provides rigidity to at least a portion of the leadless pacemaker 100, such as rigidity to the piezoelectric device 106. The housing 102 may include one or more components of the leadless pacemaker 100 that are distinct from the piezoelectric device 106. In an example, the housing 102 may include a rigid structure (e.g., one or more walls) defining an interior region. In such an example, one or more components of the leadless pacemaker 100 may be disposed in the interior region or attached to the walls. In an example, the housing 102 may include a rigid structure (e.g., framework) and one or more components of the leadless pacemaker 100 may be attached to the rigid structure. It is noted that the components of the housing 102 attached to the rigid structure (other than the piezoelectric device 106) may form the outer surface 104 of the housing 102 to which the piezoelectric device 106 is attached. In an example, the housing 102 includes components of the leadless pacemaker 100 attached together. Again, the components of the leadless pacemaker 100 (other than the piezoelectric device 106) that are attached together may form the outer surface 104 of the housing 102 to which the piezoelectric device 106 is attached. Due to the variety of structures and arrangements of the components that may form the housing 102 (as discussed above), the housing 102 is illustrated as only including the energy storage device 107. That said, the housing 102 may include additional components.

In an example, the housing 102 of the leadless pacemaker 100 includes an energy storage device 107. The energy storage device 107 may include a battery (e.g., a rechargeable battery), one or more capacitors, any other suitable energy storage device 107, or combinations thereof. The energy storage device 107 is configured to store electrical energy therein. The electrical energy of the energy storage device 107 is configured to provide electrical power to one or more components of the leadless pacemaker 100. For example, the electrical energy of the energy storage device 107 may be configured to provide electrical power to at least one electrode (e.g., the anode 108 and the cathode 110) that sends electrical pulses into the heart, one or more sensors, or electrical circuitry. The energy storage device 107 may be configured to be rechargeable by the piezoelectric device 106 which allows the energy storage device 107 to last longer. The energy storage device 107 may include sufficient electrical energy that, without the piezoelectric device 106, the energy storage device 107 can power the leadless pacemaker 100 under typical operating conditions for about 4 or more years, such as about 5 or more years, about 6 or more years, about 7 or more years, about 8 or more years, about 9 or more years, about 10 or more years, about 11 or more years, about 12 or more years, about 13 or more years, about 14 or more years, about 15 or more years, or in ranges of about 4 years to about 6 years, about 5 years to about 7 years, about 6 years to about 8 years, about 7 years to about 9 years, about 8 years to about 10 years, about 9 years to about 11 years, about 10 years to about 12 years, about 11 years to about 13 years, about 12 years to about 14 years, or about 13 years to about 15 years. For example, the energy storage device 107 may exhibit a capacity of about 75 mAh or greater, such as in ranges of about 75 mAh to about 125 mAh, about 100 mAh to about 150 mAh, about 125 mAh to about 175 mAh, about 150 mAh to about 200 mAh, about 175 mAh to about 225 mAh, about 200 mAh to about 250 mAh, about 225 mAh to about 275 mAh, or about 250 mAh to about 300 mAh. It is noted that the piezoelectric device 106 may allow the energy storage device 107 to exhibit a battery capacity (and thus a volume) that is less than that used in conventional leadless pacemakers.

The housing 102 may include one or more sensors. Examples of the sensors that may be included in the housing 102 may include one or more of activity sensors (accelerometers); pressure sensors; body temperature sensors, sensors that detect ATP levels, electrical sensors configured to detect characteristics of the heart, the energy generated by the piezoelectric device 106, or the electrical pulses provided from the electrodes to the heart; sensors configured to detect one or more characteristics of the energy storage device 107; or any other suitable sensors. It is noted that the sensors may be at least partially distinct and separate from the housing 102, for example, when the sensors are attached to or extend through an outer surface 112 of the piezoelectric device 106 or the sealant 130.

The housing 102 may include electrical circuitry. The electrical circuitry may include at least one processor and memory (e.g., non-transitory memory) storing one or more operational instructions that are configured to be executed by the processor. The electrical circuity is configured to control one or more components of the leadless pacemaker 100. For example, the electrical circuity may direct when the electrodes provide electrical pulses into the heart and the power of the electrical pulses provided by the electrodes. In an example, the electrical circuity may control one or more components of the leadless pacemaker 100 responsive to the sensors detecting one or more characteristics. In an example, the electrical circuity may include or be connected to a transceiver. The transceiver allows the electrical circuity to communicate with one or more devices external to the leadless pacemaker 100, such as one or more devices disposed in the individual (implanted sensors or other implanted devices) or one or more devices outside of the individual. The transceiver may allow the electrical circuity to provide information (e.g., the characteristics sensed by the sensors) or receive information or instructions from the external devices.

The leadless pacemaker 100 includes at least one electrode that is configured to provide electrical pulses to the heart which, in turn, can treat one or more conditions of the heart. The at least one electrode may include at least one anode 108 and at least one cathode 110. One or more of the electrodes may form part of the housing 102 or, as illustrated, may be separate from the housing 102 (e.g., disposed on the outer surface 112 of the piezoelectric device 106 or on the sealant 130). The at least one electrode may exhibit any suitable shape, such as an annular shape (as shown). It is noted that the electrode may not extend from the rest of the leadless pacemaker 100 or may only extend from the rest of the leadless pacemaker 100 for a distance equal to or less than a thickness of the electrode.

The housing 102 may include at least one attachment element 114. The attachment element 114 is configured to be attached to the device that positions the leadless pacemaker 100 in the heart of the individual (e.g., the catheter or a probe). In an example, as shown, the attachment element 114 includes a bulbous structure that may be gripped by the device that positions the leadless pacemaker 100. In an example, the attachment element 114 may include another structure instead of or in addition to the bulbous structure, such as a magnet.

The housing 102 may include at least one securement element 116 that is configured to secure the housing 102 to the heart after positioning the leadless pacemaker 100 in the heart of the individual. In an example, as shown, the housing 102 may include one or more times that are configured to secure the leadless pacemaker 100 in the heart of the individual. In an example, the housing 102 may include one or more passageways or other structures that allows the leadless pacemaker 100 to be stitched to the heart tissue or other tissue surrounding the heart. In an example, the housing 102 may include any other suitable securement element or combinations of any of the securement elements disclosed herein.

In an embodiment, at least a portion of the housing 102 (e.g., the portion of the housing 102 that is enclosed by the piezoelectric device 106) may exhibit a generally dumbbell-like shape. It is currently believed that the dumbbell-like shape of the housing 102 allows the piezoelectric device 106 to generate more electrical energy (thereby increasing the lifespan of the leadless pacemaker 100 by a longer period of time) than if the housing 102 exhibited another shape, as shown in the working examples. The generally dumbbell-like shape may include two end portions 118 that are longitudinally spaced from each other along the longitudinal axis 120 of the leadless pacemaker 100. The two end portions 118 may exhibit a relatively disc-like shape when the leadless pacemaker 100 exhibits a generally cylindrical shape. The generally dumbbell-like shape of the housing 102 may also include an intermediate portion 122 extending between the two end portions 118. The intermediate portion 122 may be rigid to maintain the position of the two end portions 118 relative to each other and to maintain the general shape of the leadless pacemaker 100. The intermediate portion 122 generally exhibits a shape that corresponds to the general shape of the leadless pacemaker 100 to allow a gap 124 having relatively constant thickness to be present between the piezoelectric device 106 and the intermediate portion 122.

The gap 124 provides a location for a portion of the piezoelectric device 106 to deform into. For example, without the gap 124, the piezoelectric device 106 would be substantially unable to deflect towards the housing 102 when the pressure applied to the piezoelectric device 106 is increased and may only deflect away from the housing 102 when the pressure decreases. In such an example, the ability of the piezoelectric device 106 to generate electrical energy is significantly dampened. However, the gap 124 allows the piezoelectric device 106 to deform towards the housing 102 when the pressure applied to the piezoelectric device 106 is increased. Allowing the piezoelectric device 106 to deform inwardly significantly increases the electrical energy generated by the piezoelectric device 106.

As previously discussed, the gap 124 is formed at least because the intermediate structure 122 exhibits a lateral dimension that is less than the corresponding lateral dimension of the two end portions 118. A maximum thickness of the gap 124 measured perpendicularly to the longitudinal axis 120 (e.g., the difference in the corresponding lateral dimensions of the intermediate structure 122 and at least one of the two end portions 118) may be selected to be about 75 um or greater, such as about 100 μm or greater, about 200 μm or greater, about 300 μm or greater, about 400 μm or greater, about 500 μm or greater, or in ranges of about 75 μm to about 150 μm, about 100 μm to about 200 μm, about 150 μm to about 250 μm, about 200 μm to about 300 μm, about 250 μm to about 350 μm, about 300 μm to about 400 μm, about 350 μm to about 450 μm, about 400 μm to about 500 μm, about 450 μm to about 550 μm, or about 500 μm to about 600 μm. Generally, the gap 124 is selected to be as small as possible to maximize the volume of the housing 102 (e.g., maximize the size of the energy storage device 107) while also minimizing the size of the leadless piezoelectric device 106. That said, the gap 124 should be selected to be large enough to prevent the piezoelectric device 106 from contacting the housing 102 or barely allow the piezoelectric device 106 to contact the housing 102 during typical operating pressures such that the intermediate portion 122 does not affect deformation of the piezoelectric device 106. The amount that the piezoelectric device 106 may deform during use depends on the flexibility of the piezoelectric device 106 which, in turn, depends on the materials forming the piezoelectric device 106, the thicknesses of the layers of the piezoelectric device 106, and the number of layers that form the piezoelectric device 106. As such, the gap 124 may be selected based on these factors.

The piezoelectric device 106 is configured to generate electrical energy during use. For example, the piezoelectric device 106 includes at least one piezoelectric material 126 that is configured to generate electrical energy responsive to being deformed. The piezoelectric device 106 includes one or more electrical contacts attached to the piezoelectric material 126. The electrical contacts electrically couple the piezoelectric device 106 to the energy storage device 107. As such, the electrical energy generated by the piezoelectric device 106 may recharge the energy storage device 107.

It is currently believed that the electrical energy generated by the piezoelectric device 106 may be sufficient to provide at least about 10% of the electrical energy needed to operate the leadless pacemaker 100, such as at least about 15%, at least about 20%, at least about 25%, at least about 30%, at least about 40%, at least about 50%, at least about 60%, at least about 70%, at least about 80%, or about 10% to about 20%, about 15% to about 25%, about 20% to about 30%, about 25% to about 40%, about 30% to about 50%, about 40% to about 60%, about 50% to about 70%, about 60% to about 80%, or about 70% to about 90% of the electrical energy needed to operate the leadless pacemaker 100. In a particular example, the electrical energy generated by the piezoelectric device 106 may not be able to generate all the electrical energy needed to power the leadless pacemaker 100, for instance, due to the limited size of the piezoelectric device 106, the efficiency of currently available biocompatible piezoelectric materials, and the electrical energy needed to power current leadless pacemakers 106. As such, in an embodiment, the piezoelectric device 106 is merely configured to recharge the energy storage device 107 rather than directly power the leadless pacemaker 100. That said, in some embodiments, the piezoelectric device 106 may directly power the leadless pacemaker 100, for instance, to give the energy storage device 107 time to cool down.

The piezoelectric device 106 may include a plurality of layers. FIG. 1D is an enlarged cross-sectional view of the piezoelectric device 106 taken from the box 1D shown in FIG. 1C. The piezoelectric device 106 includes a piezoelectric material 126. The piezoelectric material 126 may include any piezoelectric material. For example, the piezoelectric material 126 may include at least one of a piezoelectric polymer (e.g., polyvinylidene fluoride, polyvinylidene fluoride-based polymer blends and/or composites, polyvinylidene fluoride-co-trifluoroethylene, or poly (L-lactic acid)), lead zirconate titanate, barium titanate, lead titanate, gallium nitride, zinc oxide, ferroelectrics, or any other piezoelectric material. In an example, the piezoelectric material may include a biocompatible piezoelectric material.

In a particular example, the piezoelectric material 126 may include polyvinylidene fluoride (“PVDF”). PVDF is a piezoelectric polymer that is biocompatible and exhibits relatively high piezoelectric properties. PVDF may also be formed into composites (e.g., nanocomposites) and/or copolymers which allow the piezoelectric properties to be tailored to the specific individual or group of individuals while maximizing the electrical energy generated by the PVDF piezoelectric material 126. The PVDF is also a relatively flexible piezoelectric material 126 thereby allowing the piezoelectric device 106 including PVDF to exhibit relatively large deformations when exposed to the lower pressures in the right ventricle of the heart. In an embodiment, the PVDF piezoelectric material 126 may not include electrospun PVDF. In an embodiment, the PVDF piezoelectric material 126 may not include electrospun PVDF since it is believed that the electrospun PVDF is susceptible to leaks which may cause the piezoelectric device 106 to short and it is difficult to form electrical contacts on the electrospun PVDF.

The piezoelectric material 126 may exhibit a thickness measured perpendicular to the longitudinal axis 120 that is about 50 μm to about 750 μm, such as in ranges of about 50 μm to about 100 μm, about 75 μm to about 125 μm, about 100 μm to about 150 μm, about 125 μm to about 175 μm, about 150 μm to about 200 μm, about 175 μm to about 250 μm, about 200 μm to about 300 μm, about 250 μm to about 350 μm, about 300 μm to about 400 μm, about 350 μm to about 450 μm, about 400 μm to about 500 μm, about 450 μm to about 600 μm, or about 500 μm to about 700 μm. It is noted that the thickness of the piezoelectric material 126 refers to the thickness of one layer of the piezoelectric material 126 and not the collective thicknesses of the layers of the piezoelectric material 126 when the piezoelectric device 106 includes a plurality of layers of the piezoelectric material 126. The thickness of the piezoelectric layer 126 is selected to be as small as possible since decreasing the thickness of the piezoelectric layer 126 increases the deflection of the piezoelectric layer 126 at a given pressure and decreases the overall thickness of the piezoelectric device 106 which, in turn, decreases the size of the leadless pacemaker 100. Furthermore, decreasing the thickness of the piezoelectric material 126 allows the piezoelectric device 106 to include more layers of the piezoelectric material 126. However, the minimum thickness of the piezoelectric material 126 may be limited by manufacturing constraints and the composition of the piezoelectric material 126 (e.g., the strength of the material forming the piezoelectric material 126 needs to be able to withstand the forces applied thereto during operation over a prolonged period of time).

The piezoelectric device 106 may include an insulator material 128 disposed between adjacent layers of the piezoelectric material 126 when the piezoelectric device 106 includes a plurality of layers of the piezoelectric material 126 and, optionally, between the piezoelectric material 126 and the housing 102. The insulator material 128 prevents shorts between the piezoelectric material 126 and between the piezoelectric material 126 and the housing 102 which may damage or otherwise impede the operation of the leadless pacemaker 100. The insulator material 128 may also limit or prevent fluids from entering the piezoelectric device 106.

The insulator material 128 may include any suitable insulator material. In an example, the insulator material 128 may include a biocompatible insulator material. In an example, the insulator material 128 may include at least one of an epoxy, polyethylenimine, silicone elastomers (e.g., polydimethylsiloxane), or another suitable insulator. In an example, the insulator material 128 may also function as an adhesive configured to attach the different layers of the piezoelectric device 106 together and attach the piezoelectric device 106 to the housing 102. Selecting the insulator material 128 to also function as an adhesive may reduce the number of layers needed to form the piezoelectric device 106 (thereby decreasing the size of the piezoelectric device 106 or allowing the piezoelectric device 106 to include more layers) since the piezoelectric device 106 would not require a separate adhesive layer.

In a particular example, the insulator layer 128 may include a silicone elastomer. The silicone insulator material 128 may be a more beneficial insulator material than at least some other insulator materials for a variety of reasons. For example, silicone elastomers are well known biocompatible materials. Silicone also acts as an adhesive and is an effective sealant due to the hydrophobicity thereby which prevents fluids from flowing into the piezoelectric device 106. The silicone insulator material 128 is also a relatively flexible material such that the silicone insulator material 128 may negligibly affect the deformation of the piezoelectric device 106.

The insulator material 128 may exhibit a thickness measured perpendicular to the longitudinal axis 120 that is about 10 μm to about 20 μm, about 15 μm to about 30 μm, about 20 μm to about 40 μm, about 30 μm to about 50 μm, about 40 μm to about 70 μm, about 50 μm to about 100 μm, about 75 μm to about 125 μm, about 100 μm to about 150 μm, about 125 μm to about 175 μm, about 150 μm to about 200 μm, about 175 μm to about 250 μm, about 200 μm to about 300 μm, or about 300 μm or greater. It is noted that the thickness of the insulator material 128 refers to the thickness of one layer of the insulator material 128 and not the collective thicknesses of the layers of the insulator material 128 when the piezoelectric device 106 includes a plurality of layers of the insulator material 128. The thickness of the insulator material 128 is generally selected to be as small as possible since decreasing the thickness of the insulator material 128 increases the deflection of the piezoelectric device 106 at a given pressure and decreases the overall thickness of the piezoelectric device 106 which, in turn, decreases the size of the leadless pacemaker 100. Furthermore, decreasing the thickness of the insulator material 128 allows the piezoelectric device 106 to include more layers of the piezoelectric material 126. However, the minimum thickness of the insulator material 128 may be limited by manufacturing constraints, the composition of the insulator material 128 (e.g., the strength of the material forming the insulator material 128 needs to be able to withstand the forces applied thereto during operation over a prolonged period of time), and the desire to minimize any dielectric effect between the adjacent layers of the piezoelectric material 126.

In an embodiment, as shown, the piezoelectric material 126 and the insulator material 128 exhibit a spiral arrangement. The spiral arrangement of the piezoelectric material 126 and the insulator material 128 allows the piezoelectric material 126 to include a plurality of adjacent layers along a radial direction while also allowing each the piezoelectric material 126 and the insulator material 128 to be formed from a single sheet of material.

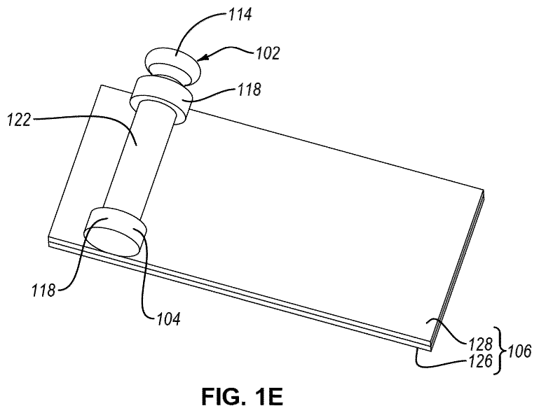

The spiral arrangement of the piezoelectric material 126 and the insulator material 128 facilitates formation of the leadless pacemaker 100. For example, FIG. 1E is an isometric view of a method of forming the leadless pacemaker 100 when the piezoelectric material 126 and the insulator material exhibit the spiral arrangement, according to an embodiment. As shown in FIG. 1E, the piezoelectric material 126 and the insulator material 128 may be provided as single sheets. Other materials (not shown) may also be provided, such as at least one adhesive material when the insulator material 128 does not also function as an adhesive. The insulator material 128 may be positioned on the piezoelectric material 126 and the housing 102 may be positioned on the insulator material 128. The housing 102, the piezoelectric material 126, and the insulator material 128 may then be rolled up to form the leadless pacemaker 100. The method illustrated in FIG. 1E may be simpler to form than if the piezoelectric material 126 and the insulator material 128 were formed from a plurality of distinct layers that are concentrically positioned relative to each other (as shown in FIG. 3).

The spiral arrangement of the piezoelectric material 126 and the insulator material 128 also facilitate formation of the leadless pacemaker 100 by minimizing the number of electrical contacts that need to be formed on the piezoelectric material 128. For example, it may be difficult to connect electrical contacts to the piezoelectric material 128 due to the relatively small thickness thereof. The spiral arrangement of the piezoelectric material 126 requires only two electrical contacts be connected thereto to electrically couple the piezoelectric material 128 to the energy storage device 107. Meanwhile, each layer of a piezoelectric material including a plurality of distinct and concentrically arranged piezoelectric materials (as shown in FIG. 3) require two electrical contacts be connected thereto.

The piezoelectric device 106 may include any suitable number of layers of the piezoelectric material 126. For example, the piezoelectric device 106 may include 1, 2, 3, 4 (as shown in FIGS. 1B-1D), 5, 6, 7, 8, 9, 10, or more than 10 layers of the piezoelectric material 126. It is noted that the layers refer to the number of layers of the piezoelectric material 126 taken along an axis that is perpendicular to the longitudinal axis 120 (e.g., a radial direction) that are adjacent to each other and are not separated from each other by the housing 102. As such, the piezoelectric device 106 may include a plurality of layers of the piezoelectric material 126 even though the piezoelectric material 126 is formed from a single sheet. The number of layers of the piezoelectric material 126 effects the electrical energy generated by the piezoelectric device 106. Increasing the number of layers of the piezoelectric material 126 increases the surface area of the piezoelectric material 126. Generally, increasing the surface area of the piezoelectric material 126 increases the electrical energy generated by the piezoelectric device 106. However, increasing the number of layers of the piezoelectric material 126 also increases the thickness of the piezoelectric device 106 which, in turn, decreases the flexibility of the piezoelectric device 106. Generally, decreasing the flexibility of the piezoelectric device 106 decreases the electrical energy generated by the piezoelectric device 106. As such, the number of layers of the piezoelectric material 126 in the piezoelectric device 106 is selected based on balancing the desire to increase the surface area of the piezoelectric material 126 while maintaining the piezoelectric device 106 sufficiently flexible. The piezoelectric device 106 may exhibit an optimal number of layers of the piezoelectric material 126. The optimal number of layers of the piezoelectric material 126 depends on several factors, such as the thickness of the piezoelectric material 126, the insulator material 128, and the optional adhesive layer; the composition (e.g., the Young's modulus) of the piezoelectric material 126, the insulator material 128, and the optional adhesive layer; etc. For example, the optimal number of layers for a piezoelectric device 106 including a PVDF piezoelectric material 126 is generally 2-5 layers while the optimal number of layers for a piezoelectric device 106 including a more rigid piezoelectric material 126 is generally 1-3 layers.

The outermost surface of each layer of the piezoelectric material 126 may exhibit a surface area of about 150 mm² to about 4,000 mm², such as in ranges of about 150 mm² to about 400 mm². about 200 mm² to about 500 mm², about 250 mm² to about 750 mm², about 500 mm² to about 1000 mm², about 750 mm² to about 1250 mm², about 1000 mm² to about 1500 mm², about 1250 mm² to about 1750 mm², about 1500 mm² to about 2000 mm², about 1750 mm² to about 2250 mm², about 2000 mm² to about 2500 mm², about 2250 mm² to about 3000 mm², about 2500 mm² to about 3500 mm², or about 3000 mm² to about 4000 mm². It is noted that each layer of the piezoelectric material 126 exhibiting the spiral arrangement refers to the portion of the piezoelectric material 126 that completes one revolution around the housing 102. Since the piezoelectric material 126 may exhibit a plurality of layers, the total surface area of the outermost surface of the piezoelectric material 126 may be any of the above surface areas multiplied by the number of layers of the piezoelectric material 126. Generally, increasing the surface area of the outer surface of the piezoelectric layer increases the electrical energy generated by the piezoelectric device 106. However, the surface area of the piezoelectric device 106 is limited by the size of the leadless pacemaker 100.

The piezoelectric device 106 may exhibit an overall thickness that is about 1 mm or less, such as about 900 μm or less, about 800 μm or less, about 700 μm or less, about 600 μm or less, about 500 μm or less, about 400 μm or less, about 300 μm or less, about 200 μm or less, or in ranges of about 150 μm to about 300 μm, about 200 μm to about 400 μm, about 300 μm to about 500 μm, about 400 μm to about 600 μm, about 500 μm to about 700 μm, about 600 μm to about 800 μm, about 700 μm to about 900 μm, or about 800 μm to about 1 mm. The overall thickness of the piezoelectric device 106 depends on the thickness of the layers thereof and the number of layers that form the piezoelectric device 106. As such, the overall thickness of the piezoelectric device 106 may be selected for any of the reasons discussed above regarding the thicknesses of the piezoelectric material 126 and the insulator material 128. The overall thickness of the piezoelectric device 106 may also be selected to keep the overall thickness of the leadless pacemaker 100 within a range that allows the leadless pacemaker 100 to be delivered to the heart and be disposed within the heart, as previously discussed.

In an embodiment, the leadless pacemaker 100 includes sealant 130 disposed on at least a portion of the exposed outer surfaces of the leadless pacemaker 100. For example, the leadless pacemaker 100 may include the sealant 130 disposed on the outer surface 112 of the piezoelectric device 106 and on the portions of the outer surface 104 of the housing 102 that are not covered by the piezoelectric device 106. The sealant 130 protects the components of the leadless pacemaker 100 from fluids, such as blood. For example, exposing the piezoelectric material 126 to blood may cause the piezoelectric device 106 to short and exposing the housing 102 to blood may cause oxidation of the housing 102. The sealant 130 may also prevent the material of the leadless pacemaker 100 from entering the blood.

The sealant 130 may include any suitable sealant material. In an example, the sealant 130 may include a hydrophobic and biocompatible material. In an example, the sealant 130 may include at least one of an epoxy or a silicone elastomer. In a particular example, the sealant 130 may include a silicone elastomer. As previously discussed, silicone is a biocompatible material that exhibits good flexibility. Also, the silicone sealant 130 may also function effectively with a silicone insulator material 128, for example, since the silicone insulator material 128 and the silicone sealant 130 exhibit the same or at least similar properties.

The sealant 130 exhibits a thickness measured perpendicular to the longitudinal axis 120. The thickness of the sealant 130 may be about 10 μm or more, such as about 20 μm or more, about 30 μm or more, about 40 μm or more, about 50 μm or more, about 75 μm or more, about 100 μm or more, about 125 μm or more, about 150 μm or more, about 200 μm or more, about 250 μm or more, about 300 μm or more, or in ranges of about 10 μm to about 30 μm, about 20 μm to about 40 μm, about 30 μm to about 50 μm, about 40 μm to about 75 μm, about 50 μm to about 100 μm, about 75 μm to about 125 μm, about 100 μm to about 150 μm, about 125 μm to about 200 μm, about 150 μm to about 250 μm, or about 200 μm to about 300 μm. The thickness of the sealant 130 is generally minimized to minimize the effect that the sealant 130 has on the flexibility of the piezoelectric device 106 and to minimize the volume of the leadless pacemaker 100. That said, the thickness of the sealant 130 should be thick enough that the sealant 130 is unlikely to crack as the piezoelectric device 106 continuously deforms during operation. It is currently believed that a silicone sealant 130 may be able to exhibit a thickness as low as 20 μm without developing cracks during normal operation of the leadless pacemaker 100.

It is noted that one or more components of the leadless pacemaker 100 may be disposed on or extend through the sealant 130. In an example, the at least one electrode (e.g., the anode 108 and the cathode 110) may be disposed on or extend through the sealant 130 to allow the at least one electrode to provide electrical pulses to the heart. In an example, sensors may be disposed on or extend through the sealant 130.

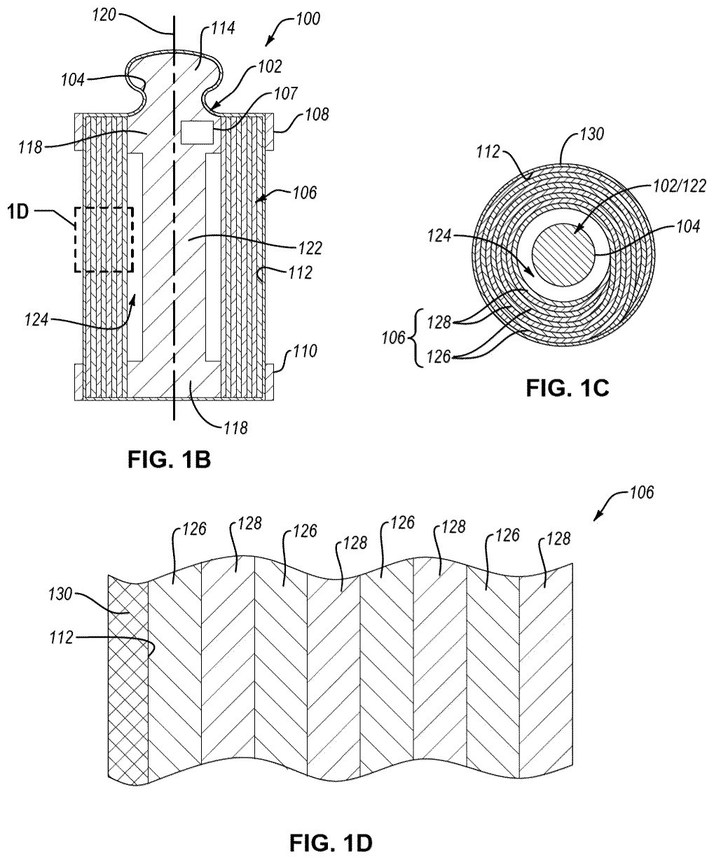

FIG. 2 is a cross-sectional view of a leadless pacemaker 200, according to an embodiment. Except as otherwise disclosed herein, the leadless pacemaker 200 is the same as or substantially similar to any of the leadless pacemakers disclosed herein. For example, the leadless pacemaker 200 includes a housing 202 having an outer surface 204 and a piezoelectric device 206 disposed on at least a portion of the outer surface 204 of the housing 202. The leadless pacemaker 200 may also include at least one electrode 210 and a sealant 230. For simplicity, the individual components of the housing 202 and the piezoelectric device 206 are not shown.

The housing 202 includes two end portions 218 that are longitudinally spaced from each other along the longitudinal axis 220 of the leadless pacemaker 200. The two end portions 218 are not attached together using a rigid intermediate structure. Instead, the housing 202 may include a flexible intermediate portion 222 extending between the two end portions 218. The flexible intermediate portion 222 may include, for example, a wire electrically and/or communicably coupling the two end portions 218 together.

The two end portions 218 may exhibit larger volumes that the two end portions 118 of FIGS. 1A-1E since the housing 202 does not include a rigid intermediate portion to hold or otherwise support the components of the housing 202. The two end portions 218 may exhibit a larger volume by increasing the lateral dimensions thereof relative to the two end portions 118 or by increasing a length of the two end portions 218 relative to the two end portions 118, wherein the lateral dimensions and the length are measured perpendicular and parallel, respectively, to the longitudinal axis 220. For example, each of the two end portions 118 may exhibit a length of about 0.5 mm to about 15 mm while each of the two end portions 218 may exhibit a length of about 2 mm to about 25 mm.

The housing 202 provides additional opportunities for the piezoelectric device 206 to deform relative to the housing 102 illustrated in FIGS. 1A-1E. For example, the housing 202 may define a larger gap 224 than the housing 102 since the housing 202 does not include a rigid intermediate structure. The larger gap 224 may allow the piezoelectric device 206 to exhibit relatively large deformations, such as when the piezoelectric device 206 is relatively flexible (e.g., is formed from a flexible material, is relatively thin, and includes one or a few layers). The housing 202 is also more likely to non-negligibly deform when a shear load is applied thereto (e.g., the two side portions 218 move sideways in opposite directions relative to each other) thereby providing another means for the piezoelectric device 206 to deform. That said, as demonstrated in the working examples, it has been found that the housing 102 exhibiting a dumbbell-like shape having a rigid intermediate portion generates more electrical energy than the housing 202.

FIG. 3 is a cross-sectional view of a leadless pacemaker 300, according to an embodiment. Except as otherwise disclosed herein, the leadless pacemaker 300 is the same or substantially similar to any of the leadless pacemakers disclosed herein. For example, the leadless pacemaker 300 includes a housing 302 having an outer surface 304 and a piezoelectric device 306 disposed on at least a portion of the outer surface 304 of the housing 302. The leadless pacemaker 300 may also include at least one electrode (not shown) and a sealant 330. For simplicity, the individual components of the housing 302 are not shown.

The piezoelectric device 306 exhibits a concentric arrangement of the layers thereof. For example, the piezoelectric device 306 includes a plurality of layers of the piezoelectric material 326. Each layer of the piezoelectric material 326 is distinct from each other and is concentrically arranged relative to each other. For instance, each of the plurality of piezoelectric materials 326 may exhibit an annular shape (e.g., hollow cylindrical shape) that, when assembled, are positioned concentrically relative to each other. Similarly, the layers disposed between the layers of the piezoelectric material 326 (e.g., the insulator material 328) are also distinct from each other and are arranged concentrically.

The concentric arrangement of the layers of the piezoelectric material 326 may allow the piezoelectric device 306 to include two different types of piezoelectric materials. For example, one of the layers of the piezoelectric material 326 may include a first piezoelectric material and another layer of the piezoelectric material 326 may include a second piezoelectric material that is different from the first piezoelectric material. The different piezoelectric materials may allow the piezoelectric device 306 to exhibit more configurations that improve the piezoelectric device 306. In an example, the first and second piezoelectric materials may include different PVDF materials, such as a nanocomposite PVDF material and a PVDF copolymer. In an example, the outer layer of the piezoelectric material 326 may include a more biocompatible piezoelectric material (e.g., a biocompatible piezoelectric material having lower piezoelectric properties) while an inner layer of the piezoelectric material 326 may include a less biocompatible piezoelectric material (e.g., a less biocompatible piezoelectric material having higher piezoelectric properties), such as a piezoelectric material including lead. In such an example, the outer layer of the biocompatible piezoelectric material 326 protects and isolates the inner layer of piezoelectric material such that the inner layer of the piezoelectric material 326 is unlikely to contact or otherwise contaminate the blood.

The concentric arrangement of the layers of the piezoelectric device 306 may complicate the manufacturing of the leadless pacemaker 300. For example, the piezoelectric device 306 may be assembled by disposing each layer within each other and/or coating each layer of the piezoelectric device 306 with the next layer, both of which may be more complicated and time consuming than merely rolling up the materials. Furthermore, each layer of the piezoelectric device 306 may need at least two electrical contacts formed thereon. However, the concentric arrangement of the layers of the piezoelectric device 306 may limit damage caused by fluids leaking into the piezoelectric device 306. For example, fluids leaking between two adjacent layers of the piezoelectric material 326 may short and damage those two adjacent layers of the piezoelectric material 326 but the other layers of the piezoelectric material 326 may remain undamaged. Furthermore, each of the concentric layers of the piezoelectric device 306 may form a barrier that prevents further leakage into other layers of the piezoelectric device 306.

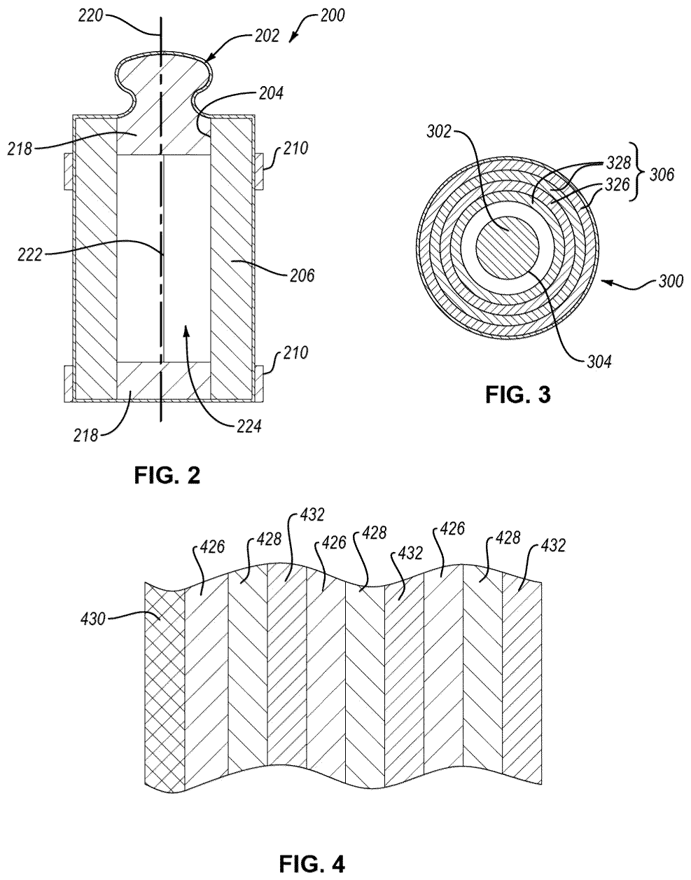

The piezoelectric devices discussed above include a piezoelectric material and an insulator material. However, the piezoelectric devices disclosed herein may include one or more additional layers. For example, FIG. 4 is a cross-sectional view of a portion of a piezoelectric device 406 with a sealant 430 disposed thereon, according to an embodiment. Except as otherwise disclosed herein, the piezoelectric device 406 may be the same as or substantially similar to any of the piezoelectric devices disclosed herein. For example, the piezoelectric device 406 may include a piezoelectric material 426 forming one or more layers and an insulator material 428 forming one or more layers.

The piezoelectric device 406 includes one or more additional layers that are distinct and separate from the piezoelectric material 426 and the insulator material 428. For example, the piezoelectric device 406 may include at least one adhesive material 432 when the insulator material 428 does not also function as an adhesive. The adhesive material 432 may include, for example, a JB weld, another epoxy adhesive, a silicone elastomer, or any other suitable adhesive.

The adhesive material 432 may exhibit a Young's modulus and a thickness measured perpendicularly to the longitudinal axis of the leadless pacemaker that generally maintains the mechanical flexibility of the piezoelectric device 406. For example, a thickness of the adhesive material 432 is about 10 μm to about 20 μm, about 15 μm to about 30 μm, about 20 μm to about 40 μm, about 30 μm to about 50 μm, about 40 μm to about 70 μm, about 50 μm to about 100 μm, about 75 μm to about 125 μm, about 100 μm to about 150 μm, about 125 μm to about 175 μm, about 150 μm to about 200 μm, about 175 μm to about 250 μm, about 200 μm to about 300 μm, or greater than 300 μm. It is noted that the thickness of the adhesive material 432 refers to the thickness of one layer of the adhesive material 432 and not the collective thicknesses of the layers of the adhesive material 432 when the piezoelectric device 406 includes a plurality of layers of the adhesive material 432. The thickness of the adhesive material 432 is generally selected to be as small as possible since decreasing the thickness of the adhesive material 432 increases the deflection of the adhesive material 432 at a given pressure and decreases the overall thickness of the piezoelectric device 406 which, in turn, decreases the size of the leadless pacemaker. Furthermore, decreasing the thickness of the adhesive material 432 allows the piezoelectric device 106 to include more layers of the piezoelectric material 426. However, the minimum thickness of the adhesive material 432 may be limited by manufacturing constraints, the composition of the adhesive material 432 (e.g., the strength of the material forming the adhesive material 432 needs to be able to withstand the forces applied thereto during operation over a prolonged period of time), and the desire to minimize any dielectric effect between the adjacent layers of the piezoelectric material 426.

The following comparative example and working examples set forth various embodiments of the leadless pacemakers disclosed herein.

Comparative example 1 was formed from a single sheet of PVDF piezoelectric material, a single sheet of epoxy insulative material disposed on the PVDF piezoelectric material, and a JB weld disposed on the epoxy insulative material. The PVDF piezoelectric material exhibited a thickness of 100 μm. The JB weld formed an adhesive layer to secure the single sheet of PVDF piezoelectric material and the single sheet of epoxy insulative material together. The JB weld also formed an inner sealant layer to prevent shorts forming in comparative example 1. Comparative example I was formed by rolling the materials together without using a housing. The hollow interior of comparative example 1 exhibited a diameter of about 6.7 mm. An epoxy sealant was disposed on the exterior surfaces of comparative example 1. Electrical contacts were made on the PVDF piezoelectric material.

Working example I was formed using the same materials and methods as comparative example 1 except that two cylindrical end portions were disposed on opposing ends of the sheets before rolling everything together. The two cylindrical end portions exhibited a diameter of about 6.7 mm. Working example 1 exhibited a cross-sectional shape that was similar to the leadless pacemaker 200 shown in FIG. 2.

Working example 2 was formed using the same materials and methods as comparative example 1 except that a dumbbell-shaped structure was disposed on top of the sheets before rolling everything together. The dumbbell-shaped structure exhibited a circular cross-sectional shape and exhibited a maximum diameter of about 6.7 mm. Working example 2 exhibited a cross-sectional shape that was similar to the leadless pacemaker 200 shown in FIG. 1B and 1C.

The testing system used to evaluate comparative example 1 and working examples 1 and 2 included a testing container having an inlet and an outlet, a fluid reservoir, and a pump. The system included a first tube extending from the inlet to the pump, a second tube extending from the pump to the fluid reservoir, and a third tube extending from the outlet of the testing container to the fluid reservoir. The testing system was configured to mimic the operations of the heart. For example, the pump was configured to, every second, remove water fluid the fluid reservoir and flow the water into the testing container to increase the pressure inside the testing container while the water may flow out of the testing container to decrease the pressure inside the testing container, thereby mimicking the operation of a heart beating at 60 bpm. The testing system includes a pressure sensor in the testing container to ensure that the pressures in the testing container are similar to the pressures that a leadless pacemaker experiences in the heart (e.g., 4-6.5 kPa). The testing system also includes electric sensors configured to detect the electrical energy generated. Each of comparative example 1, working example 1, and working example 2 were individually disposed in the testing container for testing.

When disposed in the testing container and exposed to periodic pressure increases, each of comparative example 1, working example 1, and working example 2 generated a consistent alternating current corresponding to the variations of pressure that these examples were exposed to. The consistent alternative current demonstrated that each of comparative example 1, working example 1, and working example 2 were functioning. It was noted that the consistent alternative current broke down overtime. It is believed that the breakdown of the alternating current was caused by water leaking into the structure of the examples due to a poor sealant layer being formed on the samples.

It was determined that comparative example 1 generated 0.45 nW of electrical energy, working example 1 generated 10.33 nW of electrical energy, and working example 2 generated 52.47 nW of electrical energy. This result was surprising since comparative example 1 was expected to allow the most deformation of the piezoelectrical material during operation, while working example 2 was expected to allow the least deformation of the piezoelectric material during operation. It is currently believed that the additional structure provided to the piezoelectric material by the dumbbell-shaped structure facilitates generating electrical energy with the piezoelectric material.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or ±2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

What is claimed is:

1. An energy-harvesting leadless pacemaker, comprising:
a housing including at least one outer surface, the housing including at least one energy storage device;
at least one electrode coupled to the at least one energy storage device, the at least one electrode configured to generate electrical pulses that are delivered to one or more chambers of a heart; and
at least one piezoelectric device disposed on at least a portion of the at least one outer surface of the housing, the at least one piezoelectric device electrically coupled to the at least one energy storage device, the at least one piezoelectric device configured to generate electrical energy responsive to pressure changes in the heart.

2. The energy-harvesting leadless pacemaker of claim 1, wherein the housing includes two end portions, the two end portions spaced longitudinally from each other along a longitudinal axis of the energy-harvesting leadless pacemaker.

3. The energy-harvesting leadless pacemaker of claim 2, wherein the housing includes a rigid intermediate portion extending between the two end portions.

4. The energy-harvesting leadless pacemaker of claim 3, the rigid intermediate structure exhibits a maximum lateral dimension that is less than a maximum lateral dimension of the two end portions.

5. The energy-harvesting leadless pacemaker of claim 1, wherein the at least one piezoelectric device exhibits a generally cylindrical shape.

6. The energy-harvesting leadless pacemaker of claim 1, wherein the piezoelectric material includes polyvinylidene fluoride.

7. The energy-harvesting leadless pacemaker of claim 6, wherein the polyvinylidene fluoride includes nanocomposites including at least one of polyvinylidene fluoride or copolymers including polyvinylidene fluoride.

8. The energy-harvesting leadless pacemaker of claim 1, wherein a portion of the at least one piezoelectric device and a corresponding portion of the at least one outer surface define at least one gap therebetween.

9. The energy-harvesting leadless pacemaker of claim 8, wherein the at least one gap exhibits a thickness that is about 100 μm to about 400 μm.

10. The energy-harvesting leadless pacemaker of claim 1, wherein the at least one piezoelectric device includes 2 to 5 layers of piezoelectric material between the at least one outer surface of the housing and an adjacent outer surface of the piezoelectric device.

11. The energy-harvesting leadless pacemaker of claim 1, wherein the at least one piezoelectric device includes one or more layers of piezoelectric material exhibiting a thickness of 75 μm to about 500 μm.

12. The energy-harvesting leadless pacemaker of claim 1, wherein the at least one piezoelectric device includes a single sheet of piezoelectric material exhibiting a spiral arrangement.

13. The energy-harvesting leadless pacemaker of claim 1, wherein the at least one piezoelectric device includes an insulator material between adjacent layers of piezoelectric material.

14. The energy-harvesting leadless pacemaker of claim 1, wherein the insulator material includes at least one silicone elastomer.

15. The energy-harvesting leadless pacemaker of claim 1, wherein the at least one piezoelectric device includes layer of adhesive material between adjacent layers of piezoelectric material.

16. The energy-harvesting leadless pacemaker of claim 1, further comprising a sealant coating at least an exterior surface of the at least one piezoelectric device.

17. The energy-harvesting leadless pacemaker of claim 16, wherein the at least one piezoelectric device includes an insulator material between adjacent layers of piezoelectric material, and wherein the insulator material is compositionally the same as the sealant coating.

18. The energy-harvesting leadless pacemaker of claim 16, wherein the sealant coating includes at least one silicone elastomer.

19. The energy-harvesting leadless pacemaker of claim 16, wherein the sealant coating exhibits a thickness of about 20 μm to about 100 μm.

20. A method of using an energy-harvesting leadless pacemaker, the method comprising:
disposing the energy-harvesting leadless pacemaker in a heart, the energy-harvesting leadless pacemaker including:
a housing including at least one outer surface, the housing including at least one energy storage device;
at least one electrode coupled to the at least one energy storage device, the at least one electrode configured to generate electrical pulses that are delivered to one or more chambers of the heart; and
at least one piezoelectric device disposed on at least a portion of the at least one outer surface of the housing, the at least one piezoelectric device electrically coupled to the at least one energy storage device, the at least one piezoelectric device configured to generate electrical energy responsive to pressure changes in the heart; and
generating the electrical energy with the at least one piezoelectric device as the heart beats.

21. The method of claim 20, wherein disposing the energy-harvesting leadless pacemaker in a heart includes disposing the energy-harvesting leadless pacemaker in the right ventricle of the heart.

22. A method of making an energy-harvesting leadless pacemaker, the method comprising:
disposing at least one piezoelectric device on at least one outer surface of a housing, the housing including at least one energy storage device;
wherein the energy-harvesting leadless pacemaker further includes:
at least one electrode coupled to the at least one energy storage device, the at least one electrode configured to generate electrical pulses that are delivered to one or more chambers of a heart; and
at least one piezoelectric device disposed on at least a portion of the at least one outer surface of the housing, the at least one piezoelectric device electrically coupled to the at least one energy storage device, the at least one piezoelectric device configured to generate electrical energy responsive to pressure changes in the heart.
